(19) United States
(12) Patent Application Publication
(10) Pub. No.: US 2011/0300214 A1
(43) Pub. Date: Dec. 8, 2011

(54) PHARMACEUTICAL COMPOSITIONS COMPRISING 5-CHLORO-N-(-METHYL)-2-THIOPHENCARBOXAMID

(75) Inventors: Katrin Rimkus, Iserlohn (DE); Frank Muskulus, Laupheim (DE); Sandra Brueck, Ottenhofen (DE); Jana Paetz, Bonn (DE)

(73) Assignee: RATIOPHARM GMBH, ULM (DE)

(21) Appl. No.: 13/002,972

(22) PCT Filed: Jul. 7, 2009
(86) PCT No.: PCT/EP09/04911

§ 371 Date: Aug. 23, 2011

(30) Foreign Application Priority Data
Jul. 8, 2008 (EP) 08012304.5

(51) Int. Cl. A61K 9/14; A61K 9/28; B05D 5/00; A61P 9/10; B05D 1/02; A61K 31/5377; A61P 9/00

(52) U.S. Cl. 424/465; 514/230.8; 424/490; 424/489; 427/2.14

(57) ABSTRACT

The invention relates to pharmaceutical compositions comprising 5-Chloro-N-({(5S)-2-oxo-3-[4-(3-oxo-4-morpholinyl)-phenyl]-1,3-oxazolidin-5-yl}-methyl)-2-thiophencarboxamid and processes of preparing such compositions. In a second aspect, the present invention relates to a preferred pellet-layering process for preparing such compositions.

The invention relates to pharmaceutical compositions comprising 5-Chloro-N-({(5S)-2-oxo-3-[4-(3-oxo-4-morpholinyl)-phenyl]-1,3-oxazolidin-5-yl}-methyl)-2-thiophencarboxamid suitable for immediate release and processes of preparing such compositions.

5-Chloro-N-({(5S)-2-oxo-3-[4-(3-oxo-4-morpholinyl)-phenyl]-1,3-oxazolidin-5-yl}-methyl)-2-thiophencarboxamid is a low-molecular, orally administrable inhibitor of the blood coagulation factor Xa, investigated for the prophylaxis and/or treatment of various thrombo-embolic diseases (see WO 01/47919) and known under the INN rivaroxaban or under the trade name Xarelto®. The 5-Chloro-N-({(5S)-2-oxo-3-[4-(3-oxo-4-morpholinyl)-phenyl]-1,3-oxazolidin-5-yl}-methyl)-2-thiophencarboxamid has the following chemical structure.

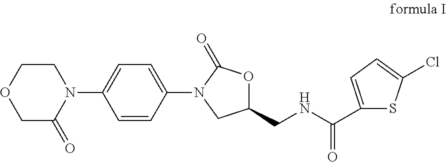

The compounds according to formula I will be hereinafter referred to as “Compound I”. In this regard it is noted that the terms “Compound I” or “compound according to formula I” refer to 5-Chloro-N-({(5S)-2-oxo-3-[4-(3-oxo-4-morpholinyl)-phenyl]-1,3-oxazolidin-5-yl}-methyl)-2-thiophencarboxamid and its solvates and hydrates as well as pharmaceutical acceptable salts thereof, preferably obtained according to the procedures as outlined in WO 01/47919. This form has been described in WO2007/039132 as crystalline form I.

In the art, several formulations of compound I are known. For example, formulations having modified release properties are described in WO 2006/072367.

Compound I has only limited solubility in water, causing problems regarding dissolution of the API from the pharmaceutical composition and the oral bioavailability.

In order to improve the bioavailability of Compound I, several concepts have been put forward. WO2005/060940 teaches the use of the wet granulation technique in combination with the use of solubilizers in order to hydrophilize the Compound I and to improve bioavailability.

WO 2007/039122 discloses immediate release forms comprising the use of an amorphous or semi-stable crystalline modification of Compound I as API. The use of these modifications significantly increases the solubility and the oral bioavailability compared to the formulations described in WO2005/060940, using the Compound I in crystalline modification I.

Employing the above hydrophilization by wet granulation approach, using the stable crystalline modification Compound I, does not provide sufficient bioavailability compared to using the amorphous state according to the teaching in WO2007/039122. The use of Compound I in the amorphous state is hampered by stability issues due to the tendency of the amorphous form to switch to a semi-crystalline state. The wet granulation technique furthermore is energy and time-consuming and cost-intensive.

It is therefore an object of the invention to provide a process for the manufacture of a pharmaceutical composition comprising Compound I or a pharmaceutically acceptable salt thereof which does not encounter the above mentioned problems. In particular, a pharmaceutical composition should be provided having improved properties like solubility, dissolution profile, stability, flowability and bioavailability. Especially, it was an object of the present invention to provide an immediate release pharmaceutical rivaroxaban composition having a superior dissolution profile even after prolonged time of storage.

Furthermore, it has been found that the content uniformity of the pharmaceutical compositions as disclosed in WO 2005/060940 is still optimizable. Particularly in the case of rivaroxaban, a superior content uniformity is desirable, since the interindividual variability in pharmacokinetics is significant and ranges from 30% to 40% (see Product Monograph Xarelto®, 2008). Therefore, it was a further object of the present invention to provide pharmaceutical compositions comprising rivaroxaban suitable for having a superior dissolution profile and a superior high content uniformity.

Immediate release oral dosage forms should be provided, wherein due to stability reasons the use of any disintegrant is reduced or even avoided.

Moreover, it has been found that the process as described in WO 2005/060940 is still optimizable with regard to operational health and safety, in particular with regard to the production of respirable dust. Hence, it was an object of the present invention to provide a process for preparing a rivaroxaban formulation, wherein the production of respirable dust is reduced or preferably completely avoided.

It has now been found that the above problems can be overcome by providing pharmaceutical formulations comprising Compound I, a solubilizer and a pseudo-emulsifier as excipients.

The problem can be further overcome by specific processes for the manufacture of a pharmaceutical formulation of Compound I or its solvates and hydrates.

Hence, a subject of the present invention is a pharmaceutical composition comprising

(a) a compound according to formula I as active ingredient

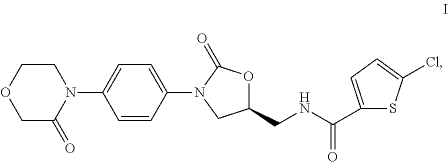

its solvates, hydrates and/or pharmaceutically acceptable salts,
(b) a solubilizer and
(c) a pseudo-emulsifier as excipients.

In the pharmaceutical composition of the present invention Compound I as the active ingredient (component (a)) preferably is present in crystalline form, wherein the crystalline modification I as described in WO2005/060940 is particularly preferred. Preferably, the active ingredient is present in the form of the free base.

In a preferred embodiment the active ingredient (a) is employed in a micronized form. That means, the active ingredient (a) of the pharmaceutical composition of the present invention (=Compound I) has a volume mean particle size (D₅₀) of 0.1 to 100 μm, more preferably of 0.3 to 50 μm, further more preferably of 1 to 20 μm, most preferably of 2 to 10 μm.

Within this application, the volume mean particle size (D₅₀) is determined by the light scattering method, using a Mastersizer 2000 apparatus made by Malvern Instruments (wet measurement, 2000 rpm, ultrasonic waves for 60 sec., data interpretation via Fraunhofer Method).

The pharmaceutical composition further comprises one or more solubilizers (b). Generally, the term “solubilizer” means any organic excipient, which improves the solubility and dissolution of the active pharmaceutical ingredient. Preferably, the solubilizer is capable of reducing the dissolution time of a pharmaceutical composition by 5%, more preferably by 20%, according to USP 31-NF26 release method, using apparatus 2 (paddle), compared to the same pharmaceutical composition comprising calcium hydrogen phosphate instead of the solubilizer.

The solubilizers are selected, for example, from the group of known inorganic or organic excipients. Such excipients preferably include polymers, low molecular weight oligomers, natural products and surfactants.

Preferably, the solubilizer is a water-soluble compound, having a water solubility of more than 10 mg/l, more preferably of more than 20 mg/l, still more preferably of more than 50 mg/l at a temperature of 25° C. The solubility of the solubilizer might be e.g. up to 1,000 mg/l or up to 300 mg/ml at a temperature of 25° C. The water-solubility is determined according to the column elution method of the Dangerous Substances Directive (67/548/EEC), Annex V, Chapter A6.

In a preferred embodiment the solubilizer is a hydrophilic polymer, preferably having the above mentioned water-solubility. Generally, the term “hydrophilic polymer” encompasses polymers comprising polar groups. Examples for polar groups are hydroxy, amino, carboxy, carbonyl, ether, ester and sulfonate. Hydroxy groups are particularly preferred.

The hydrophilic polymer usually has a weight average molecular weight, ranging from 1,000 to 250,000 g/mol, preferably from 2,000 to 100,000 g/mol, particularly from 4,000 to 50,000 or 4,000 to 70,000 g/mol. Furthermore, a 2% w/w solution of the hydrophilic polymer in pure water preferably has a viscosity of from 1 to 8 mPas or 2 to 8 mPas at 25° C. The viscosity is determined according to the European Pharmacopoeia (hereinafter referred to as Ph. Eur.), 6^{th} edition, chapter 2.2.10.

Furthermore, the hydrophilic polymer used as solubilizer preferably has a glass transition temperature (T_{g}) or a melting point of 25° C. to 150° C., more preferably of 40° C. to 100° C. The glass transition temperature, T_{g}, is the temperature at which the hydrophilic polymer becomes brittle on cooling and soft on heating. That means, above T_{g}, the hydrophilic polymers become soft and capable of plastic deformation without fracture. The glass transition temperature or the melting point are determined with a Mettler-Toledo® DSC 1, wherein a heating rate of 10° C. per minute and a cooling rate of 15° C. per minute is applied. The determination method essentially is based on Ph. Eur. 6.1, section 2.2.34. For the determination of T_{g} the polymer is heated twice (i.e. heated, cooled, heated).

More preferably, derivatives of cellulose (hydroxypropylmethyl cellulose (HPMC), hydroxypropyl cellulose (HPC), carboxymethyl cellulose (CMC), preferably sodium or calcium salts thereof, microcrystalline cellulose, hydroxyethyl cellulose), polyvinyl-pyrrolidone, preferably having a weight average molecular weight of 10,000 to 60,000 g/mol, copolymers of polyvinylpyrrolidones, preferably copolymers comprising vinylpyrrolidone and vinylacetate units (e.g. Povidon VA 64; BASF), preferably having a weight average molecular weight of 40,000 to 70,000 g/mol, polyoxyethylene-alkylethers, polyethylene glycol, sugar alcohols like isomalt, sorbitol or mannitol, co-blockpolymers of ethylene oxide and propylene oxide (Poloxamer, Pluronic®), derivates of methacrylates, polyvinylalcohol, derivates of glycerol, derivates of polyethylene glycols, derivates of dextrines, and derivates of fatty acids, e.g. sodium lauryl sulfate, are used as solubilizers.

In particular, cellulose derivatives (especially hydroxypropylmethyl cellulose (HPMC) and/or hydroxypropyl cellulose (HPC)), sugar alcohols (especially isomalt), polyvinylpyrrolidone and copolymers of polyvinylpyrrolidone, in particular copolymers comprising vinylpyrrolidone and vinylacetate units, are used as solubilizer.

It is particularly preferred that the above mentioned kinds of hydrophilic polymers fulfill the functional requirements (molecular weight, viscosity, T_{g}, melting point, non-semi-permeable properties) as illustrated above.

In the pharmaceutical composition of the present invention at least one of the above-mentioned solubilizers is present. Alternatively, a combination of two or more solubilizers can be employed.

The pharmaceutical composition further comprises one or more pseudo-emulsifiers (c). Generally, the term “pseudo-emulsifier” means any organic excipient, which avoids an agglomeration of a micronized active ingredient (API) after disintegration of the pharmaceutical composition, in order to improve the solubility of the active ingredient.

The pseudo-emulsifiers preferably are selected from natural products, more preferably from natural gums. Natural gums are polysaccharides of natural origin, capable of causing a viscosity increase in solution, even at concentrations less than 15%. Generally, the addition of 5 wt. % (abbreviation for “weight percent”) of the pseudo-emulsifiers—preferably of the natural gum—to an aqueous solution causes a viscosity increase of said solution of at least 1%, preferably of at least 2%, especially of at least 5%. The viscosity is determined according to the European Pharmacopoeia (hereinafter referred to as Ph. Eur.), 6^{th} edition, chapter 2.2.10.

Examples for suitable natural gums are
Agar (E406), preferably obtained from seaweed,
Alginic acid (E400), preferably obtained from seaweed,
Beta-glucan, preferably from obtained oat or barley bran,
Carrageenan (E407), preferably obtained from seaweed,
Chicle gum, preferably obtained from the chicle tree,
Dammar gum, preferably obtained from the sap of Dipterocarpaceae trees,
Gellan gum (E418), preferably produced by bacterial fermentation,
Glucomannan (E425), preferably obtained from the konjac plant,
Gum arabica (E414), preferably obtained from the sap of acacia trees,
Gum ghatti, preferably obtained from the sap of Anogeissus trees,
Gum tragacanth (E413), preferably obtained from the sap of Astragalus shrubs,
Karaya gum (E416), preferably obtained from the sap of sterculia trees,
Locust bean gum (E410), preferably obtained from the seeds of the carob tree,
Mastic gum, preferably obtained from the mastic tree,
Psyllium seed husks, preferably obtained from the Plantago plant,
Sodium alginate (E401), preferably obtained from seaweed,
Spruce gum, preferably obtained from spruce trees,
Tara gum (E417), preferably obtained from the seeds of the tara tree.

Furthermore, the pseudo-emulsifier can be selected from phospholipids, preferably lecithin. Moreover, the pseudo-emulsifier can comprise proteins, preferably phosphoproteins like casein.

In a preferred embodiment the pseudo-emulsifier comprises gum arabica, agar and/or lecithin, in particular gum arabica. However, corn starch, croscarmellose, microcrystalline cellulose and Klucel® HXF are preferably not regarded as pseudo-emulsifier in the sense of the present application. Furthermore, the pseudo-emulsifier preferably is not xanthan gum.

In the pharmaceutical composition of the present invention at least one of the above-mentioned pseudo-emulsifiers is present. Alternatively, a combination of two or more pseudo-emulsifiers can be employed.

Preferred combinations of solubilizer and pseudo-emulsifier are:
Polyvinylpyrrolidone/gum arabica,
polyvinylpyrrolidone, sodium lauryl sulfate/gum arabica,
copolymers of polyvinylpyrrolidone/gum arabica,
copolymers of polyvinylpyrrolidone, sodium lauryl sulfate/gum arabica,
hydroxypropylmethyl cellulose (HPMC)/gum arabica
copolymers of polyvinylpyrrolidone and HPMC/gum arabica,
hydroxypropyl cellulose (HPC)/gum arabica,
polyvinylpyrrolidone/agar,
copolymers of polyvinylpyrrolidone/agar,
copolymers of polyvinylpyrrolidone, sodium lauryl sulfate/agar,
hydroxypropylmethyl cellulose (HPMC)/agar,
copolymers of polyvinylpyrrolidone and HPMC/agar,
hydroxypropyl cellulose (HPC)/agar,
polyvinylpyrrolidone/lecithin,
copolymers of polyvinylpyrrolidone/lecithin,
hydroxypropylmethyl cellulose (HPMC)/lecithin,
copolymers of polyvinylpyrrolidone and HPMC/lecithin,
hydroxypropyl cellulose (HPC)/lecithin,
isomalt/gum arabica,
isomalt/agar,
isomalt/lecithin, and/or
isomalt/carrageenan.

Generally, in the pharmaceutical composition of the present invention the active ingredient (a) can be present in an amount of 1 to 99 wt. %, preferably 4 to 60 wt. %, more preferably 5 to 40 wt. %, and particularly preferred between 6 and 20 wt. %, based on the total weight of the composition.

Generally, in the pharmaceutical composition of the present invention the solubilizer (b) can be present in an amount of 0.1 to 80 wt. %, preferably 0.5 to 60 wt. % or 1 to 60 wt. %, more preferably 5 to 30 wt. %, based on the total weight of the composition.

Generally, in the pharmaceutical composition of the present invention the pseudo-emulsifier (c) can be present in an amount of 0.01 to 15 wt. %, preferably 0.1 to 10 wt. %, more preferably 0.2 to 5 wt. % or 0.5 to 5 wt. %, in particular 0.5 to 2.5 wt. % or 0.8 to 2.5 wt. %, based on the total weight of the composition. It has been found that a higher amount of pseudo-emulsifier in the composition might result in an incomplete drug release. Therefore, it is preferred that the pharmaceutical composition of the present invention does not comprise more than 15 wt. % of pseudo-emulsifier, more preferably not more than 10 wt. %, particularly not more than 5%. Especially it is preferred that the pharmaceutical composition of the present invention does not comprise more than 15 wt. % of a natural gum, more preferably not more than 10 wt. %, particularly not more than 5%.

In a preferred embodiment the pharmaceutical composition of the present invention comprises
(a) the compound according to formula I in crystalline form,
(b) cellulose or derivatives thereof or polyvinylpyrrolidone or copolymers thereof as solubilizer, and
(c) a natural gum as pseudo-emulsifier.

During the dissolution of the formulation, the combination of a solubilizer and a pseudo-emulsifier usually is aimed to reduce the agglomeration of the particles during the dissolution and increase the effect of the solubilizers. The mechanism of action of the pseudo-emulsifier usually mainly relies on an enhancement of viscosity. However pseudo-emulsifiers also possess emulsifying properties.

The pharmaceutical composition of the present invention can be prepared by specific processes.

In a first embodiment the pharmaceutical composition of the present invention can be prepared by a dry-granulation process.

Hence, a further subject of the present invention is a process for producing a pharmaceutical composition, comprising the steps of
(i) mixing a compound according to formula I and excipients,
(ii) dry-compaction of the mixture to give a comprimate, and
(iii) granulating the comprimate.

In step (i) the compound according to formula I (=Compound I) is mixed with excipients. The mixing process can be carried out in conventional mixers, e.g. in a free fall mixer like Turbula T 10B (Bachofen AG, Switzerland).

Preferably, the excipients comprise a solubilizer and a pseudo-emulsifier. Generally, it is noted that all comments made above regarding the solubilizer (b) and the pseudo-emulsifier (c) of the pharmaceutical composition of the present invention also apply for the processes of the present invention.

In the process of the present invention (in addition to solubilizer and pseudo-emulsifier) one or more pharmaceutically acceptable excipient(s), such as fillers, binding agents, lubricants, glidants, anti-sticking agents, and disintegrating agents, can be employed. Regarding the above-mentioned pharmaceutically acceptable excipients, the application refers to “Lexikon der Hilfsstoffe für Pharmazie, Kosmetik and angrenzende Gebiete”, edited by H. P. Fiedler, 4th Edition, Edito Cantor, Aulendorf and earlier editions, and “Handbook of Pharmaceutical Excipients”, Third Edition, edited by Arthur H. Kibbe, American Pharmaceutical Association, Washington, USA, and Pharmaceutical Press, London. In this regard it is generally noted that, due to the nature of pharmaceutical excipients, it cannot be excluded that a certain compound meets the requirements of more than one of the components (b) and (c) or of the above mentioned additional excipients. However, in order to enable an unambiguous distinction, it is preferred in the present application that one and the same pharmaceutical compound can only function as one of the compounds (b) or (c) or additional excipient. For example, if mannitol functions as solubilizer (b) it cannot additionally function as pseudo-emulsifier (c) or as filler or as binding agent. Furthermore, in the present application rivaroxaban only functions as component (a) but not as one of components (b) or (c).

Preferred examples of the fillers are soluble and insoluble excipients like lactose or calcium hydrogen phosphate. The filler is for example present in an amount of 0 to 80 wt. %, preferably of 10 to 60 wt. %, of the total weight of the composition.

The binding agent can for example be starch. Preferably, the binding agent is present in an amount of 0 to 25 wt. %, more preferably at 2 to 10 wt. %, of the total weight of the composition.

The lubricant is preferably a stearate or fatty acid, more preferably an earth alkali metal stearate, such as magnesium stearate. The lubricant is suitably present in an amount of 0 to 2 wt. %, preferably about 0.5 to 1.5 wt. % of the total weight of the composition.

Preferred disintegrating agents are croscarmellose sodium, sodium carboxymethyl starch, cross-linked polyvinylpyrrolidone (crospovidone) or sodium carboxymethyl glycolate (e.g. Explotab®), sodium bicarbonate. The disintegrating agent is suitably present in an amount of 0 to 20 wt. %, more preferably at about 1 to 15 wt. % of the total weight of the composition.

The glidant can for example be colloidal silicon dioxide (e.g. Aerosil®). Preferably the glidant agent is present in an amount of 0 to 8 wt. %, more preferably at 0.1 to 3 wt. % of the total weight of the composition.

The anti-sticking agent is for example talcum and may be present in amounts of 0 to 5% .wt, more preferably in an amount of 0.5 to 3 wt. % of the total weight of the composition.

Generally, if in the processes of the present invention solubilizers (b) or pseudo-emulsifiers (c) are used, all other excipients (e.g. fillers, binding agents, lubricants, disintegrating agents, glidants and anti-sticking agents) are defined as not comprising those compounds which were specified above as being solubilizers or pseudo-emulsifiers.

In the second step (ii) the mixed formulation is subjected to a dry-compaction step in order to receive a comprimate. The dry-compaction generally is carried out in the absence of essential amounts of solvents.

In a preferred embodiment the dry-compaction step is carried out by roller compaction. Alternatively, e.g. slugging can be used. If roller compaction is applied, the compaction force usually ranges from 2 to 50 kN/cm, preferably from 5 to 45 kN/cm, more preferably from 8 to 28 kN/cm.

The gap width of the roller compactor usually is 0.8 to 5 mm, preferably 1 to 4 mm, more preferably 1.5 to 3.2 mm, especially 1.8 to 3.0 mm.

Preferably, the roller compactor is equipped with a cooling device. Usually, the comprimated pharmaceutical composition should not be subjected to temperatures above 50° C.

In a third step of the (first embodiment of the) process of the present invention (iii) the comprimate (received in step (ii)) is granulated.

Preferably, the granulation step is carried out by an elevated sieving equipment, e.g. Comil® U5 (Quadro Engineering, USA).

It is further possible, that in the process of the present invention a so-called multiple compaction is carried out. In this case the particles resulting from step (iii) are recycled into the compaction step (ii). Optionally, further excipients can be added during each cycle. Preferably, 2 to 5, more preferably 3 to 4 cycles are carried out.

In a preferred embodiment the granulation conditions are chosen such that the resulting granulated pharmaceutical composition comprises a volume mean particle size (D₅₀) of 10 to 1000 μm, more preferably of 20 to 800 μm, further more preferably of 50 to 700 μm, most preferably of 100 to 650 μm. The volume mean particle size (D₅₀) is determined by the light scattering method, using a Mastersizer 2000 apparatus made by Malvern Instruments.

The bulk density of the granulated pharmaceutical composition made by the process of the first embodiment generally ranges from of 0.2 to 0.85 g/ml, preferably of 0.25 to 0.85 g/ml, more preferably of 0.3 to 0.8 g/ml or 0.40 to 0.80 g/ml.

The granulated pharmaceutical composition of the invention made by the process of the first embodiment preferably possesses Hausner ratios in the range of 1.01 to 1.6 or 1.05 to 1.6, preferably of 1.06 to 1.4, more preferably between 1.08 to 1.3 or 1.08 to 1.25. The Hausner ratio is the ratio of tapped density to bulk density.

In a second embodiment the pharmaceutical composition of the present invention can be prepared by a pellet layering process.

Hence, a further subject of the present invention is a process for producing a pharmaceutical composition, comprising the steps of
(i) providing a pellet core,
(ii) providing a solution or suspension comprising the compound according to formula I, and
(iii) spraying the solution or suspension onto the pellet core.

In this second embodiment, the present invention provides a process for the manufacture of a pharmaceutical composition comprising Compound I, employing a pellet layering process. Herein Compound I is dispersed in a solution or dispersion of one or more pharmaceutically acceptable excipients. This solution or suspension is sprayed onto an inert core, which is made from water soluble or insoluble materials.

In step (i) a pellet core is provided. Preferably, the pellet core is a so-called neutral pellet core, that means it does not comprise an active ingredient. The pellet core can be made of suitable materials, e.g. cellulose, sucrose, starch or mannitol or combinations thereof.

More preferably solubilizers used for the pellet core are selected from derivatives of cellulose (hydroxypropylmethyl cellulose (HPMC), hydroxypropyl cellulose (HPC), hydroxyethyl cellulose), polyvinylpyrrolidone, copolymers of polyvinylpyrrolidones (Povidon VA 64; BASF), polyoxyethylene-alkylethers, polyethylene glycol, sugar alcohols like isomalt, sorbitol or mannitol, co-block polymers of ethylene oxide and propylene oxide (Poloxamer).

Suitable pellet cores are commercially available under the trade name Cellets® and preferably comprise microcrystalline cellulose. In a particular, preferred embodiment pellet cores, commercially available as Suglets®, are used. Those preferred pellet cores comprise a mixture of corn starch and sucrose. The mixture usually comprises 1 to 20 wt. % corn starch and 80 to 99 wt. % sucrose, in particular, about 8 wt. % corn starch and 92% sucrose.

In step (ii) the compound according to formula I (=Compound I) is dissolved or suspended in a solvent. The solvent can be water, a pharmaceutically acceptable organic solvent or mixtures thereof. Preferably, the solvent is water or an alcohol. Most preferably, the solvent is water.

The solution or dispersion of Compound I can comprise further excipients. It preferably comprises a solubilizer and/or a pseudo-emulsifier. Generally, it is noted that all comments made above regarding the solubilizer (b) and the pseudo-emulsifier (c) of the pharmaceutical composition of the present invention also apply for the processes of the present invention. In addition, the solution or dispersion may comprise anti-sticking agents and lubricants. Reference is made to the explanations given above for the first embodiment of the process of the present invention.

In the third step (iii) the emulsion or suspension is sprayed onto the pellet core, preferably by an fluid bed dryer, e.g. Glatt GPCG 3 (Glatt GmbH, Germany) or Innojet® Ventilus 1 (Innojet Herbert Huettlin, Germany).

In a preferred embodiment the spraying conditions are chosen such that the resulting particulate pharmaceutical composition comprises a volume mean particle size (D50) of 10 to 1000 μm, more preferably of 20 to 800 μm, further more preferably of 100 to 750 μm, most preferably of 250 to 650 μm. The volume mean particle size (D50) is determined by the light scattering method using a Mastersizer 2000 apparatus made by Malvern Instruments.

The bulk density of the particulate pharmaceutical composition made by the process of the second embodiment generally ranges from of 0.2 to 0.85 g/ml, preferably of 0.25 to 0.85 g/ml, more preferably of 0.4 to 0.85 g/ml.

The particulate pharmaceutical composition of the invention made by the process of the second embodiment preferably possesses Hausner ratios in the range of 1.05 to 1.6, preferably of 1.08 to 1.4, more preferably between 1.10 to 1.3.

In a third embodiment the pharmaceutical composition of the present invention can be prepared by a melt granulation or melt coating process, wherein Compound I preferably is dispersed with at least one solubilizer, a pseudo-emulsifier and optionally a pharmaceutically acceptable carrier or matrix by a melting (fusion) process, i.e. Compound I is granulated with a melted mass of excipients. After cooling, the obtained mass is preferably granulated, i.e. for example crunched, grinded and sieved. Alternatively, the melted mass can be charged directly in a mold to give tablets.

Hence, a further subject of the present invention is a process for producing a pharmaceutical composition, comprising the steps of
(i) mixing a compound according to formula I and excipients,
(ii) melting the mixture,
(iii) cooling off and granulating the melted mixture.

In step (i) the compound according to formula I (=Compound I) is mixed with excipients. Preferably, the excipients comprise a solubilizer and a pseudo-emulsifier. Generally, it is noted that all comments made above regarding the solubilizer (b) and the pseudo-emulsifier (c) of the pharmaceutical composition of the present invention also apply for the processes of the present invention.

Optionally, also a carrier or matrix, employing the following polymeric material, can be used: derivatives of cellulose, sugar alcohols, derivatives of organic acids, derivatives of fatty acids, waxes, semi-synthetic derivatives of glycerol.

For the melt granulation, for example, an extrusion process or high shear process may be used. The melting conditions are preferably chosen such that the active ingredient remains in crystalline form I.

In a fourth embodiment the pharmaceutical composition of the present invention can be prepared by a co-precipitation process, wherein the Compound I is dissolved together with a suitable polymer in an organic solvent. By addition of an anti-solvent a Compound I-polymer-complex is precipitated.

Hence, a further subject of the present invention is a process for producing a pharmaceutical composition, comprising the steps of
(i) dissolving a compound according to formula I and polymer excipients in a solvent,
(ii) precipitating a complex comprising a compound according to formula I and polymer excipients by adding an anti-solvent, and
(iii) granulating the precipitated complex, and optionally
(iv) adding a pseudo-emulsifier (a).

In step (i) the compound according to formula I (=Compound I) is mixed with polymer excipients. Preferably, the polymer excipients comprise a solubilizer. Generally, it is noted that all comments made above regarding the solubilizer (b) and the pseudo-emulsifier (c) of the pharmaceutical composition of the present invention also apply for the processes of the present invention.

The solvent could be a pharmaceutically acceptable organic solvent or mixtures thereof. Preferably, the solvent is an alcohol or an organic acid. Most preferably, the solvent is acetic acid or ethanol.

In the second step (ii) a complex, comprising a compound according to formula I and polymer excipients, is precipitated by adding an anti-solvent. The anti-solvent could be water or a pharmaceutically acceptable organic solvent or a mixture thereof. Preferably, the anti-solvent is water. If necessary, also a pH-shift could be employed in order to induce precipitation.

The obtained complex is granulated (that means for example crunched, grinded and sieved) in a third step, preferably by any sieving machine, e.g. Comil® U5.

In a preferred embodiment the granulation conditions are chosen such that the resulting granulated pharmaceutical composition comprises a volume mean particle size (D₅₀) of 10 to 500 μm, more preferably of 20 to 400 μm, further more preferably of 50 to 300 μm, most preferably of 50 to 200 μm. The volume mean particle size (D₅₀) is determined by the light scattering method using a Mastersizer 2000 apparatus made by Malvern Instruments.

The bulk density of the granulated pharmaceutical composition made by the process of the fourth embodiment generally ranges from of 0.2 to 0.85 g/ml, preferably of 0.25 to 0.85 g/ml, more preferably of 0.3 to 0.75 g/ml.

The granulated pharmaceutical composition of the invention made by the process of the fourth embodiment preferably possesses Hausner ratios in the range of 1.05 to 1.6, preferably of 1.08 to 1.4, more preferably between 1.10 to 1.3.

As mentioned above, four processes are suitable for preparing the pharmaceutical compositions of the present invention. Said processes lead to pharmaceutical compositions in granulate form. Therefore, a further subject of the present invention are granulates (=particles) obtainable by any of the processes of the present invention. These granules can be regarded as a so-called “primary pharmaceutical composition”.

Regarding the terms “granulates” and “granulate form”, it is noted that within this application these terms refer to any particulate form of the (primary) pharmaceutical composition. Preferably, the granules have mean diameters as mentioned above. That means, that the terms “granulates” and “granulate form” may also cover particles which are in the art sometimes referred to as “pellets”.

The granulates of the present invention (i.e. the primary pharmaceutical composition) may be used to prepare suitable solid oral dosage forms. That means, the primary pharmaceutical composition can be further processed to give a “final pharmaceutical composition”, i.e. to give a final dosage form. Preferably, the granulates can be compressed to a tablet or filled into capsules or sachets, optionally after blending with other excipients. A particularly preferred dosage form is in the form of tablets.

The dosage forms of the present invention (preferably the tablets) may contain dosage amounts of 1 to 60 mg, more preferable 10 to 50 mg, e.g. 10 mg, 20 mg, 25 mg or 50 mg of the active pharmaceutical ingredient. Thus the administered amount can be readily varied according to individual tolerance and safety warranting a flexible dosing.

Hence, a further subject of the present invention is a process for producing tablets, comprising the steps of
(i) optionally mixing the granulates of the present invention with further excipients, and
(ii) compressing the granulates of the present invention or the mixture of step (i) to give tablets.

In step (i) the granulates (the primary pharmaceutical composition) can be mixed with further excipients.

In the process for producing tablets (i.e. the final pharmaceutical composition) one or more pharmaceutically acceptable excipient(s), such as fillers, binding agents, lubricants, glidants, anti-sticking agents, and disintegrating agents, can be employed. Usually, these further excipients are added in addition to the excipients, which have already been employed in the preparation of the granulates (i.e. in the preparation of the primary pharmaceutical composition).

Preferred examples of the fillers are soluble and insoluble excipients like lactose or calcium hydrogen phosphate. As mentioned above, the filler is for example present in an amount of 0 to 80 wt. %, preferably of 10 to 60 wt. % of the total weight of the final pharmaceutical composition.

The binding agent can for example be starch. Preferably, the binding agent is present in an amount of 0 to 25 wt. %, more preferably at 2 to 10 wt. % of the total weight of the final pharmaceutical composition.

The lubricant is preferably a stearate or fatty acid, more preferably an earth alkali metal stearate, such as magnesium stearate. The lubricant is suitably present in an amount of 0 to 2 wt. %, preferably about 0.5 to 1.5 wt. % of the total weight of the final pharmaceutical composition.

Preferred disintegrating agents are croscarmellose sodium, sodium carboxymethyl starch, cross-linked polyvinylpyrrolidone (crospovidone) or sodium carboxymethyl glycolate (e.g. Explotab®), sodium bicarbonate. The disintegrating agent is suitably present in an amount of 0 to 20 wt. %, more preferably at about 1 to 15 wt. % of the total weight of the final pharmaceutical composition.

The glidant can for example be colloidal silicon dioxide (e.g. Aerosil®). Preferably the glidant agent is present in an amount of 0 to 8 wt. %, more preferably at 0.1 to 3 wt. % of the total weight of the final pharmaceutical composition.

The anti-sticking agent is for example talcum and may be present in amounts of 0 to 5% .wt, more preferably in an amount of 0.5 to 3 wt. % of the total weight of the final pharmaceutical composition.

Generally, the amounts of above-mentioned further excipients which are employed in the compression step depend on the amounts of excipients which have already been employed in the process for producing the granulates (i.e. in the process for producing the primary pharmaceutical composition). For example, if the final pharmaceutical composition should comprise 30% binder, it would be possible to add 20% binder before the compaction step and 10% binder before the compression step or e.g. alternatively 25% binder before the compaction step and 5% binder before the compression step.

The compression step (ii) is preferably carried out with a rotary press, e.g. on a Fette 102i (Fette GmbH, Germany) or a Riva® piccola (Riva, Argentina).

In an alternative embodiment, tablets comprising the pharmaceutical composition of the present invention can be prepared by a direct-compression method.

Hence, a further subject of the present invention is a process for producing tablets comprising a pharmaceutical of the present invention, comprising the steps of
(i) mixing a compound according to formula I and excipients
(ii) direct-compressing said mixture.

The excipients used in the direct compression are defined as described above and preferably contain also the solubilizer (b) and the pseudo-emulsifier (c).

The direct compression is preferably carried out on a rotary press, e.g. on a Fette 102i (Fette GmbH, Germany) or a Riva® piccola (Riva, Argentina).

If a rotary press is applied, the main compaction force usually ranges from 1 to 50 kN, preferably from 2 to 40 kN, more preferably from 2.5 to 35 kN.

Finally, subjects of the present inventions are tablets obtainable by any of the processes as described above.

The tablets of the present invention tablets can be film-coated tablets for peroral use or dispersing tablets.

The film-coating agent is for example hydroxypropylmethyl cellulose or methacrylate and may be present in an amount of 1-10 wt. %, more preferably in an amount of 2-8 wt. %, based on the total weight of the composition.

In another issue, the present invention provides the use of the pharmaceutical composition of the present invention for the prophylaxis and/or treatment of thrombo-embolic diseases, such as infarct, angina pectoris (including instable angina) re-occlusions and restenoses after an angioplasty or an aorta-coronary bypass, stroke, transitory ischaemic events, peripheral arterial occlusion, lung embolism or deep vein thrombosis.

Where it is referred to the total weight of the pharmaceutical composition and the pharmaceutical composition in a single dosage form, the total weight is the weight of the single dosage form excluding, if applicable, the weight of any coating or capsule shell.

The pharmaceutical compositions and tablets of the present invention are formulations showing “immediate release”. Within the scope of this patent application, immediate release formulations having a Q value of not less than 75%, preferably having a Q value from 80% to 100%, more preferably a Q value from 90% to 100%. The Q value is determined as described in USP 32-NF 27 method II (paddle, chapter <711>). In case of tablets this values refer to the uncoated tablet.

Furthermore, the pharmaceutical compositions and tablets of the present invention preferably do not comprise compounds imparting modified release properties. More preferably, the pharmaceutical compositions and tablets of the present invention do not comprise a modified release system comprising a non-erodible polymer and a pore-forming substance.

The above explanations illustrate the first aspect of the present invention. In addition, in a second aspect of the present invention it was unexpectedly found that a pharmaceutical rivaroxaban composition having superior properties (e.g. desirable dissolution profile and a superior content uniformity) is obtainable by the above-mentioned pellet-layering process, even if the pseudo-emulsifier (c) is not present.

Therefore, a further subject of the present invention is a pharmaceutical composition comprising
(a) a compound according to formula I as active ingredient

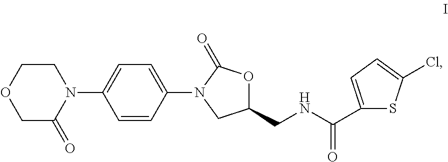

its solvates, hydrates and/or pharmaceutically acceptable salts and
(b) a solubilizer,
obtainable by a process comprising the steps of
   (i) providing a pellet core,
   (ii) providing a solution or suspension comprising the compound according to formula I (a) and a solubilizer (b), and
   (iii) spraying the solution or suspension onto the pellet core.

Generally, in the second aspect of the invention for compound I (a) the same considerations apply as given above for the first aspect.

Generally, in the second aspect of the invention for the solubilizer (b) the same considerations apply as given above for the first aspect.

Preferably, in the second aspect of the present invention the solubilizer is a water-soluble compound, having a water solubility of more than 10 mg/l, more preferably of more than 20 mg/l, still more preferably of more than 50 mg/l at a temperature of 25° C. The solubility of the solubilizer might be e.g. up to 1,000 mg/l or up to 300 mg/ml at a temperature of 25° C. The water-solubility is determined according to the column elution method of the Dangerous Substances Directive (67/548/EEC), Annex V, Chapter A6.

In a preferred embodiment the solubilizer is a hydrophilic polymer, preferably having the above mentioned water-solubility and film-building properties. Generally, the term “hydrophilic polymer” encompasses polymers comprising polar groups. Examples for polar groups are hydroxy, amino, carboxy, carbonyl, ether, ester and sulfonate. Hydroxy groups are particularly preferred.

The hydrophilic polymer usually has a weight average molecular weight, ranging from 1,000 to 250,000 g/mol, preferably from 2,000 to 100,000 g/mol, particularly from 4,000 to 70,000 g/mol. Furthermore, a 2% w/w solution of the hydrophilic polymer in pure water preferably has a viscosity of from 1.0 to 8.0, preferably 1.2 to 5.0 mPas at 25° C. The viscosity is determined according to the European Pharmacopoeia (hereinafter referred to as Ph. Eur.), 6^{th} edition, chapter 2.2.10.

Furthermore, the hydrophilic polymer used as solubilizer preferably has a glass transition temperature (T_{g}) or a melting point of 25° C. to 150° C., more preferably of 40° C. to 100° C. (wherein the definition of T_{g} is given above).

Preferred examples of suitable solubilizers are derivatives of cellulose (hydroxypropylmethyl cellulose (HPMC), hydroxypropyl cellulose (HPC), carboxymethyl cellulose (CMC), preferably sodium or calcium salts thereof, polyvinylpyrrolidone, preferably having a weight average molecular weight of 10,000 to 60,000 g/mol, copolymers of polyvinylpyrrolidones, preferably copolymers comprising vinylpyrrolidone and vinylacetate units (e.g. Povidon VA 64; BASF), preferably having a weight average molecular weight of 40,000 to 70,000 g/mol, polyoxyethylene alkylethers, polyethylene glycol, sugar alcohols like isomalt, sorbitol or mannitol, co-blockpolymers of ethylene oxide and propylene oxide (Poloxamer, Pluronic®). Especially preferred as hydrophilic polymer (=solubilizer b) is polyvinylpyrrolidone, particularly having a weight average molecular weight of from 15.000 to 35.000 g/mol.

In a further preferred embodiment the solubilizer (b) comprises two components

(b1) a hydrophilic polymer as described above; and
(b2) a surfactant,
wherein the weight ratio (b1) to (b2) usually ranges from 50:1 to 1:1, preferably 20:1 to 2:1.

Generally, surfactants are agents that lower the surface tension of a liquid. Surfactants are usually organic compounds that are amphiphilic, i.e. they contain both hydrophobic groups and hydrophilic groups.

Preferably, anionic surfactants are used as component (b2), e.g. sodium lauryl sulfate.

Hence, in a preferred embodiment the solubilizer comprises or consists of polyvinylpyrrolidone, particularly having a weight average molecular weight from 15.000 to 35.000 g/mol, and a surfactant, preferably sodium lauryl sulfate.

Generally, in the pharmaceutical composition of the second aspect of the present invention the solubilizer (b) can be present in an amount of 0.1 to 60 wt. %, preferably 0.2 to 20 wt. % or 0.3 to 10 wt. %, more preferably 0.5 to 5 wt. %, based on the total weight of the composition.

In the second aspect of the present invention the pharmaceutical composition usually is free of pseudo-emulsifiers, wherein the term “pseudo-emulsifier” is defined as above in the first aspect of the present invention. Hence, in the second aspect of the present invention the pharmaceutical composition usually is free of a natural gum.

The pharmaceutical composition of the second aspect of the present invention is prepared by a pellet-layering process.

Hence, a further subject of the present invention is a process for producing a pharmaceutical composition, comprising
(a) a compound according to formula I as active ingredient

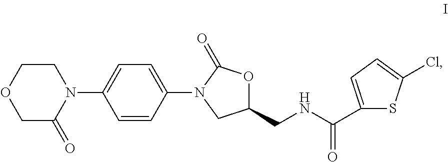

its solvates, hydrates and/or pharmaceutically acceptable salts and
(b) a solubilizer, wherein said process comprises the steps of
   (i) providing a pellet core,
   (ii) providing a solution or suspension comprising the compound according to formula I (a) and a solubilizer (b), and
   (iii) spraying the solution or suspension onto the pellet core.

In step (i) a pellet core is provided. Preferably, the pellet core is a so-called neutral pellet core, that means it does not comprise an active ingredient. The pellet core can be made of suitable materials, e.g. cellulose or derivatives (particularly hydroxypropylmethyl cellulose (HPMC), hydroxypropyl cellulose (HPC), ethyl cellulose, hydroxyethyl cellulose, sucrose, starch or mannitol or combinations thereof.

Suitable pellet cores are commercially available under the trade name Cellets® and preferably comprise microcrystalline cellulose. In a particular preferred embodiment pellet cores, commercially available as Suglets®, are used. Those preferred pellet cores comprise a mixture of corn starch and sucrose. The mixture usually comprises 1 to 20 wt. % corn starch and 80 to 99 wt. % sucrose, in particular about 8 wt. % corn starch and 92% sucrose.

Usually, the pellet cores have a volume average particle size (D50) from 200 to 600 μm, preferably from more than 250 to 500 μm, more preferably from 255 to 360 μm, particularly from 260 to 340 μm. The particle size is determined as described above.

In order to enable a clear distinction between the compounds used in the present invention, the pellet core is preferably not regarded as solubilizer (b).

In step (ii) the compound according to formula I (=Compound I=rivaroxaban) is dissolved or suspended in a solvent, preferably suspended. The solvent can be water, a pharmaceutically acceptable organic solvent, or mixtures thereof. Preferably, the solvent is water or an alcohol. Most preferably, the solvent is water.

Usually, Compound I (a) is present in the solvent in an amount of 1 to 30, preferably 5 to 20 wt. %, more preferably from 10 to 15 wt. %. Usually, the solubilizer(s) (b) is/are present in the solvent in an amount of 0.1 to 20, preferably 0.5 to 10 wt. %, more preferably from 2 to 8 wt. %.

In addition, the solution or dispersion may comprise anti-sticking agents and lubricants as described below. However, it is preferred that the solution or suspension consists of solvent, Compound I (a) and one or more solubilizers (b) and optionally an anti-sticking agent.

In the third step (iii) the emulsion or suspension is sprayed onto the pellet core (and subsequently dried), preferably in an fluid bed dryer or a fluid bed granulator, e.g. Glatt® GPCG 3 (Glatt GmbH, Germany) or Innojet® Ventilus 1 (Innojet Herbert Huettlin, Germany).

Usually, in these apparatuses the pellet cores are fluidized in a stream of gas, preferably air, and the solution or suspension prepared in step (ii) is sprayed, preferably from a nozzle, onto the bed of pellet cores. Usually, sufficient solution or suspension is sprayed to produce a coating of the desired thickness. Subsequently, usually the spray is turned off. Preferably, the fluidizing gas is continued until the coated pellets are dried in the fluidizing gas stream. Therefore, step (iii) can be regarded as a “spray-drying” step.

Hence, after spraying and drying the solution or suspension comprising compound I (a) and solubilizer(s) (b), a coating is formed on the pellet core. The coating usually has a thickness from 0.1 to 50 μm, preferably from 5.0 to 40 μm, more preferably from 15 to 35 μm, particularly from 20 to 30 μm. The thickness of the coating is determined microscopically.

Therefore, a further subject of the second aspect of the present invention is a coated pellet, comprising
(i) a pellet core, wherein the pellet core has an diameter of 100 to 600 μm, preferably from 200 to 500 μm, more preferably from 250 to 355 μm; and
(ii) a coating comprising Compound I (=rivaroxaban (a)) and solubilizer (b), wherein the coating has a thickness from 0.1 to 50 μm, preferably from 5.0 to 40 μm, more preferably from 15 to 35 μm, particularly from 20 to 30 μm.

The diameter of the pellet core is determined microscopically and defined by its longest dimension.

Generally, in the second aspect of the present invention the pellet core is free of active agent, i.e. free of rivaroxaban.

The bulk density of the particulate pharmaceutical composition made by the process of the second aspect (or of the coated pellets as described above) generally ranges from 0.2 to 0.95 g/ml, preferably from 0.40 to 0.82 g/ml, more preferably from 0.45 to 0.80 g/ml.

The particulate pharmaceutical composition of the invention made by the process of the second aspect (or of the coated pellets as described above) preferably possesses Hausner ratios in the range of 1.05 to 1.6, preferably of 1.08 to 1.3, more preferably between 1.10 to 1.25.

The coated pellets as resulting from the process of the second aspect of the present invention are regarded as a “primary pharmaceutical composition”. Said primary pharmaceutical composition may be used to prepare suitable solid oral dosage forms.

That means, the primary pharmaceutical composition can be further processed to give a “final pharmaceutical composition”, i.e. to give a final dosage form, particularly an oral dosage form. Preferably, the coated pellets can be compressed to a tablet or filled into capsules or sachets, optionally after blending with other excipients. A particularly preferred dosage form is in the form of tablets.

The dosage forms of the second aspect of the present invention (preferably the tablets) may contain dosage amounts of 1 to 60 mg, more preferable 10 to 50 mg, e.g. 10 mg, 20 mg, 25 mg or 50 mg of the active pharmaceutical ingredient.

Hence, a further subject of the second aspect of the present invention is a process for producing oral dosage forms, comprising the steps of
(i) optionally mixing the coated pellets of the second aspect of the present invention with one or more further excipients, and
(ii) transferring the coated pellets or the mixture from step (i) into an oral dosage form, e.g. by filling into capsules or sachets, or alternatively by compressing into tablets.

Preferably, the oral dosage form is a tablet. Hence, a further subject of the second aspect of the present invention is a process for producing tablets, comprising the steps of
(i) mixing the coated pellets of the second aspect of the present invention with one or more further excipients, and
(ii) compressing the mixture of step (i) to give tablets.

In step (i) the coated pellets are mixed with further excipients.

In the process for producing oral dosage forms (i.e. the final pharmaceutical composition), preferably tablets, one or more pharmaceutically acceptable excipient(s), such as fillers, binders, lubricants, glidants, anti-sticking agents, and disintegrating agents, can be employed.

Fillers (or also referred to in the art as diluents) usually are added to form dosage forms of a size, suitable for handling. Preferred examples of the fillers are lactose or calcium hydrogen phosphate. The filler is usually present in an amount of 0 to 60 wt. %, preferably of 1 to 40 wt. %, more preferably 2 to 30 wt. %, still more preferably 2 to 25 wt. %, based on the total weight of the dosage form, i.e. the final pharmaceutical composition. In case of tablets, these values refer to the uncoated tablet.

A binding agent may be added to ensure that oral dosage forms, preferably tablets, can be formed with the required mechanical strength. The binding agent can for example be starch or microcrystalline cellulose. Usually, the binding agent is present in an amount of 0 to 35 wt. %, preferably of 1 to 30 wt. %, more preferably of 2 to 25 wt. %, still more preferably of 3 to 20 wt. %, based on the total weight of the final pharmaceutical composition. In case of tablets these values refer to the uncoated tablet.

The function of the lubricant is to ensure that tablet formation and ejection can occur with low friction between the solid and the die wall. The lubricant is preferably a stearate or fatty acid, more preferably an earth alkali metal stearate, such as magnesium stearate. The lubricant is suitably present in an amount of 0 to 2 wt. %, preferably of about 0.1 to 1.0 wt. % of the total weight of the final pharmaceutical composition. In case of tablets, these values refer to the uncoated tablet.

A disintegrant is a compound which enhances the ability of the dosage form, preferably the ability of the tablet, when in contact with a liquid, preferably water, to break up into smaller fragments. Preferred disintegrating agents are croscarmellose sodium, sodium carboxymethyl starch, cross-linked polyvinylpyrrolidone (crospovidone) or sodium carboxymethyl glycolate (e.g. Explotab®), or sodium bicarbonate. The disintegrating agent is suitably present in an amount of 0 to 20 wt. %, more preferably of about 1 to 15 wt. % of the total weight of the final pharmaceutical composition. In case of tablets, these values refer to the uncoated tablet.

A glidant is a compound, capable of improving the flowability of the ((primary or final) pharmaceutical composition. The glidant can for example be colloidal silicon dioxide (e.g. Aerosil®). Preferably, the glidant agent is present in an amount of 0 to 5 wt. %, more preferably of 0.1 to 2 wt. % of the total weight of the final pharmaceutical composition. In case of tablets, these values refer to the uncoated tablet.

An anti-sticking agent is a compound, capable of reducing adhesion between the particles of the pharmaceutical composition and the punch faces and thus capable of preventing particles sticking to the punches. The anti-sticking agent is for example talcum and may be present in amounts of 0 to 5 wt. %, more preferably of 0.01 to 1 wt. %, still more preferably in an amount of 0.02 to 0.5 wt. % of the total weight of the final pharmaceutical composition. In case of tablets, these values refer to the uncoated tablet.

The compression step (ii) is preferably carried out with a rotary press, e.g. on a Fette® 102i (Fette GmbH, Germany) or a Riva® piccola (Riva, Argentina). If a rotary press is applied, the main compaction force usually ranges from 1 to 50 kN, preferably from 2 to 40 kN, more preferably from 5 to 25 kN.

Hence, subjects of the second aspect of the present invention are dosage forms, particularly tablets, obtainable by any of the processes as described above.

The oral dosage forms of the second aspect of the present invention, preferably in form of tablets, usually comprise or consist of

40 to 100 wt. %, more preferably 60 to 95 wt. %, still more preferably 70 to 90 wt. % coated pellets,
0 to 40 wt. %, more preferably 2 to 30 wt. % filler,
0 to 35 wt. %, more preferably at 2 to 20 wt. % binding agent,
0 to 5 wt. %, more preferably 0.1 to 2 wt. % glidant,
0 to 3 wt. %, more preferably 0.01 to 0.5 wt. % anti-sticking agent,
0 to 2 wt. %, preferably about 0.1 to 1.0 wt. % lubricant,
0 to 20 wt. %, preferably at about 1 to 10 wt. % disintegrant,
wherein all numbers are based on the total weight of the oral dosage form. In case of tablets, these values refer to the uncoated tablet.

Alternatively, coated pellets, as described above, preferably obtained by the process as described above, can be filled into suitable containers like capsules, sachets, stick packs or the like. The coated pellets can be filled into the containers without adding further additives. Preferably, the coated pellets are blended with an anti-sticking agent and subsequently filled into the containers. Hence, the oral dosage forms of the second aspect of the present invention when filled in suitable containers, like capsules or sachets or stick-packs, usually comprise 99 to 100 wt. %, more preferably 99.5 to 99.99 wt. % coated pellets, and 0 to 1 wt. %, more preferably 0.01 to 0.05 wt. %, anti-sticking agent.

The dosage forms, preferably the tablets of the second aspect of the present invention, usually have a content uniformity of 85 to 115%, preferably of 95 to 105%, more preferably of 96 to 104%, still more preferably of 97 to 103%, particularly preferred of 98 to 102% and most preferred from 99% to 101%. The content uniformity is determined according the European Pharmacopeia (Ph. Eur), 4^{th} edition, 2002, section 2.9.6.

The tablets of the second aspect of the present invention tablets can be film-coated tablets for peroral use or dispersing tablets. The film-coating agent is for example hydroxypropylmethyl cellulose or methacrylate and may be present in an amount of 1 to 10 wt. %, more preferably in an amount of 2 to 8 wt. %, based on the total weight of the composition. Preferably, a film, not imparting modified-release properties, is used.

The pharmaceutical compositions, oral dosage forms (preferably tablets) of the second aspect of the present invention, are formulations showing “immediate release”. Within the scope of this patent application, the immediate release formulations have a Q value of not less than 75%, preferably a Q value from 80% to 100%, more preferably a Q value from 90% to 100%. The Q value is determined as described in USP 32-NF 27 method II (paddle, chapter <711>). In case of tablets this values refer to the uncoated tablet.

The pharmaceutical compositions, oral dosage forms (preferably tablets) of the second aspect of the present invention, preferably do not comprise compounds imparting modified release properties. More preferably, the pharmaceutical compositions, oral dosage forms (preferably tablets) of the second aspect of the present invention, do not comprise a modified release system comprising a non-erodible polymer and a pore-forming substance.

As summary, the essential items of the second aspect of the present invention are illustrated below.

### Item 1:

Process for producing a pharmaceutical composition, comprising
(a) a compound according to formula I as active ingredient

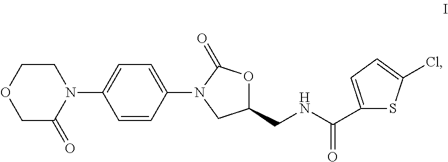

its solvates, hydrates and/or pharmaceutically acceptable salts and
(b) a solubilizer,
   wherein said process comprises the steps of
(i) providing a pellet core,
(ii) providing a solution or suspension comprising the compound according to formula I (a) and a solubilizer (b), and
(iii) spraying the solution or suspension onto the pellet core.

### Item 2:

Process according to item 1, wherein the solubilizer comprises a hydrophilic polymer.

### Item 3:

Process according to item 2, wherein the solubilizer comprises

(b1) a hydrophilic polymer; and
(b2) a surfactant,
wherein the weight ratio (b1) to (b2) preferably ranges from 50:1 to 1:1, more preferably from 20:1 to 2:1.

### Item 4:

Process according to item 3, wherein (b1) is povidone and (b2) is an anionic surfactant, preferably sodium lauryl sulfate.

### Item 5:

Process according to any one of items 1 to 4, wherein the process is carried out in the absence of a pseudo emulsifier.

### Item 6:

Process according to any one of items 1 to 5, wherein the pellet cores have a volume average particle size (D50) from 200 to 600 μm, preferably from more than 250 to 500 μm, more preferably from 255 to 360 μm, particularly from 260 to 340 μm.

### Item 7:

Process according to any one of items 1 to 6, wherein in step (iii) a coating, comprising compound I (a) and solubilizer(s) (b), is formed on the pellet core.

### Item 8:

Process according to item 7, wherein the coating has a thickness from 0.01 to 20 μm, preferably from 0.1 to 10 μm, more preferably from 1.0 to 5.0 μm, particularly from 2.0 to 4.0 μm.

### Item 9:

Pharmaceutical composition obtainable by a process according to any one of items 1 to 8.

### Item 10:

Coated pellet, comprising

(i) a pellet core, wherein the pellet core has an diameter of 100 to 600 μm, preferably from 200 to 500 μm, more preferably from 250 to 355 μm; and
(ii) a coating comprising Compound I (=rivaroxaban (a)) and solubilizer (b).

### Item 11:

Coated pellet according to item 10, wherein the coating (ii) has a thickness from 0.1 to 50 μm, preferably from 5.0 to 40 μm, more preferably from 15 to 35 μm, particularly from 20 to 30 μm.

### Item 12:

Coated pellet according to items 10 or 11, wherein the pellet core is free of Compound I and preferably consists of cellulose or derivatives thereof, or a mixture of corn starch and sucrose.

### Item 13:

An oral dosage form comprising the pharmaceutical composition according to item 8, or coated pellets according to any one of items 10 to 12, and optionally further excipients, preferably selected from fillers, binders, lubricants, glidants, anti-sticking agents, and disintegrating agents.

### Item 14:

An oral dosage form, preferably in form of a tablet, according to item 13, comprising 40 to 100 wt. %, more preferably 60 to 95 wt-%, still more preferably 70 to 90 wt. % coated pellets according to any of items 10 to 12,

0 to 40 wt. %, more preferably 2 to 30 wt. % filler,
0 to 35 wt. %, more preferably 2 to 20 wt. % binding agent,
0 to 5 wt. %, more preferably 0.1 to 2 wt. % glidant,
0 to 3 wt. %, more preferably 0.01 to 0.5 wt. % anti-sticking agent,
0 to 2 wt. %, preferably about 0.1 to 1.0 wt. % lubricant,
0 to 20 wt. %, preferably about 1 to 10 wt. % disintegrant,
wherein all numbers are based on the total weight of the oral dosage form.

### Item 15:

Process for producing an oral dosage form according to item 13, comprising the steps of

(i) mixing the pharmaceutical composition according to item 8, or coated pellets according to any one of items 10 to 12, with one or more further excipients, and
(ii) compressing the mixture of step (i) to give tablets.

The invention is now illustrated in the following examples, which are not to be constructed as being limiting. The first aspect of the invention is illustrated by Examples 1 to 6, whereas Example 7 illustrates the second aspect of the present invention.

### EXAMPLES

### Example 1

### Dry-Compaction

TABLE 1

| | Amount in [mg] calculated |
|---|---|
| Ingredient | according to a single dose |
| Compound I micronized | 20 |
| Gum Arabica | 5 |
| Povidon VA 64 | 10 |
| Sodium lauryl sulfate | 1 |
| Calcium hydrogen phosphate | 66 |
| Magnesium stearate | 0.9 |
| Coll. silicon dioxide (Aerosil ®) | 0.5 |
| Explotab ® | 9.5 |
| Hydroxypropylmethyl cellulose | 2 |
| Talcum | 0.1 |
| Total | 115 |

Micronized Compound I was blended with gum Arabica, Povidon VA 64, sodium lauryl sulfate, Explotab®, 30 mg Calcium hydrogen phosphate, 0.4 mg magnesium stearate and 0.2 mg Aerosil® for 30 min in a tumble blender, for example Turbula TC 10 B. The pre-blend was compacted at 10-30 kN and was subsequently crunched towards a defined particle size of less than 1.5 mm. The compacted material was mixed with the remaining parts of calcium hydrogen phosphate and coll. silicon dioxide for 25 min in a tumble blender. Subsequently magnesium stearate was added. The final blend was mixed for 3 min and compressed on a rotary press. The tablets has a friability of less than 1% and a hardness of 50-90 N. The tablets were coated with a suspension of hydroxypropylmethyl cellulose and talcum in a pen coater.

### Example 2

### Direct-Compression

TABLE 2

| | Amount in [mg] calculated |
|---|---|
| Ingredient | according to a single dose |
| Compound I micronized | 10.0 |
| Agar | 2.0 |
| Povidon VA 64 | 25.0 |
| Sodium lauryl sulfate | 1.0 |
| Silificied microcrystalline cellulose | 60.0 |
| Magnesium stearate | 0.9 |
| Coll. silicon dioxide (Aerosil ®) | 0.4 |
| Crospovidone | 20.0 |
| Total | 118.3 |

Rivaroxaban, Povidon VA 64, sodium lauryl sulfate, crospovidone and silificied microcrystalline cellulose were blended for 10 min in a free fall blender Turbula® TB10. The remaining excipients, apart from magnesium stearate, were added and blended for 25 min. Magnesium stearate was added and blended for further 3 min. The final blend was compressed on a rotary press riva piccolo.

### Example 3

### Pellet-Layering

TABLE 3

| | Amount in [mg] calculated |
|---|---|
| Ingredient | according to a single dose |
| Compound I micronized | 20 |
| Pellets (Cellets ®) | 30 |
| Sodium lauryl sulfate | 1 |
| Gum Arabica | 2 |
| Povidon | 5 |
| Talcum | 0.2 |
| Microcrystalline cellulose | 27.5 |
| Lactose | 20 |
| Magnesium stearate | 0.9 |
| Aerosil ® | 0.4 |
| Total | 107 |

Compound I was suspended together with talcum and gum Arabica in an aqueous solution of Povidon and SDS. The placebo pellets were preheated to 38° C. in a fluid bed dryer. Subsequently the pellets were coated with the suspension using the following parameter:

Inlet temperature: 40-80° C.
Product temperature: 35-40° C.
Spray nozzle: 1-2 mm
Spray pressure 1-2 bar

After sintering at elevated temperature the pellets were blended with MCC, Lactose and Aerosil for 25 min in a tumble blender. Afterwards magnesium stearate was added and the blend was mixed for additional 3 minutes.

The final blend was compressed to tablets, which can optionally be coated (see formulation above in example 1).

### Example 4

### Pellet-Layering

TABLE 4

| | Amount in [mg] calculated |
|---|---|
| Ingredient | according to a single dose |
| Compound I micronized | 10 |
| Pellets (Suglets ®) | 200 |
| Sodium lauryl sulfate | 0.5 |
| Gum Arabica | 1.0 |
| Povidon | 2.5 |
| Talcum | 0.1 |
| Microcrystalline cellulose (MCC) | 13.75 |
| Lactose | 10.0 |
| Magnesium stearate | 0.45 |
| Aerosil ® | 0.2 |
| Total | 238.5 |

The pellets were pre-heated in an Innojet Ventilus 1 and subsequently layered by a suspension containing rivaroxaban. The suspension was made by a solution of Povidon, gum arabicum and sodium lauryl sulfate in water, in which talc and rivaroxaban were suspended. The dried pellets were blended with MCC, Lactose and Aerosil® for 25 min in a free fall mixer Turbula® TB10. Magnesium stearate was added and blended for further 3 min. The final blend was compressed on a rotary press Riva piccolo.

The in-vitro dissolution profile of a composition according to Example 4 was determined according to USP-Paddle, 900 ml acetate buffer, pH 4.5 and 0.5% sodium lauryl sulfate, 75 rpm. The results are shown in Table 5:

TABLE 5

| time [min] | mean dissolved [%] | SD* |
|---|---|---|
| 0 | 0 | 0 |
| 5 | 94.9 | 2.0 |
| 10 | 96.2 | 2.1 |
| 15 | 96.6 | 2.1 |
| 30 | 96.9 | 2.2 |
| 60 | 97.1 | 2.2 |
| 120 | 97.2 | 2.2 |

*Standard Deviation

The dissolution profile as shown in Table 5 indicates superior properties. In particular, the standard deviation is unexpectedly low, indicating a superior content uniformity. As a comparison, when determining the standard deviation of a composition according to WO 2005/060940, a significantly higher value (about 20%) for the standard deviation was found.

### Example 5

### Melt-Extrusion

TABLE 6

| Ingredient | Amount in [mg] |
|---|---|
| Compound I micronized | 20 |
| Povidon VA 64 | 40 |
| Sodium lauryl sulfate | 1 |
| Agar | 2 |
| Lactose | 30 |
| Crospovidone | 10 |
| Magnesium stearate | 0.9 |
| Aerosil ® | 0.4 |
| Total | 104.3 |

Compound I was blended with Povidon VA 64 and SDS. The blend was extruded (70-160° C.) and sieved after cooling, resulting in a defined particle size distribution between 0.8-1.5 mm.

The sieved extrudate was pre-blended with Agar-Agar for 10 min in a tumble blender and subsequently lactose, crospovidone and Aerosil were added and the mixture was blended for additional 25 min.

Magnesium stearate was added and the mixing was completed for 3 minutes. The final blend was compressed into tablets on a rotary press: tablet specification see above.

### Example 6

### Co-Precipitation

TABLE 7

| Ingredient | Amount in [mg] |
|---|---|
| Compound I micronized | 10 |
| HPC | 50 |
| Sodium lauryl sulfate | 1 |
| Agar | 2 |
| Microcellac ® | 20 |
| Explotab ® | 10 |
| Magnesium stearate | 0.9 |
| Aerosil ® | 0.4 |
| Total | 94.3 |

Compound I was dissolved with hydroxypropyl cellulose and SDS in a mixture of acetic acid and ethanol in a ratio of 9:1. Under stirring, water as anti-solvent was added. The precipitate was dried at elevated temperatures. The co-precipitate was pre-blended with agar and finally mixed with the remaining excipients. The final blend was compressed into tablets.

### Example 7

### Pellet-Layering

TABLE 8

| | Amount in [mg] calculated |
|---|---|
| Ingredient | according to a single dose |
| Compound I micronized | 10 |
| Pellets (Suglets ®) | 200 |
| Sodium lauryl sulfate | 0.5 |
| Povidon | 2.5 |
| Talcum | 0.1 |
| Microcrystalline cellulose (MCC) | 13.75 |
| Lactose | 10.0 |
| Magnesium stearate | 0.45 |
| Aerosil ® | 0.2 |
| Total | 237.5 |

The pellets were pre-heated in an Innojet® Ventilus 1 and in subsequence layered by a suspension containing rivaroxaban. The suspension was made by a solution of povidone and sodium lauryl sulfate in water, in which talcum and rivaroxaban were suspended. The dried pellets were blended with MCC, lactose and Aerosil® for 25 min in a free fall mixer Turbula® TB10. Magnesium stearate was added and blended for further 3 min. The final blend was compressed on a rotary press Riva® piccolo.

The in-vitro dissolution profile of a composition according to Example 7 was determined according to USP-Paddle, 900 ml acetate buffer, pH 4.5 and 0.5% sodium lauryl sulfate, 75 rpm. The results are shown in Table 9:

TABLE 9

| time [min] | mean dissolved [%] | SD |
|---|---|---|
| 0 | 0 | 0 |
| 5 | 94.1 | 1.9 |
| 10 | 94.5 | 2.0 |
| 15 | 95.0 | 1.9 |
| 30 | 95.7 | 2.0 |
| 60 | 95.9 | 2.0 |
| 120 | 96.2 | 2.0 |

The dissolution profile as shown in Table 9 indicates superior properties. In particular, the standard deviation is unexpectedly low, indicating a superior content uniformity. As a comparison, when determining the standard deviation of a composition according to WO 2005/060940, a significantly higher value (about 20%) for the standard deviation was found.

### Example 8

### Pellet-Layering

TABLE 10

| | Amount in [mg] calculated |
|---|---|
| Ingredient | according to a single dose |
| Compound I micronized | 10 |
| Pellets (Suglets ®) | 200 |
| Sodium lauryl sulfate | 0.5 |
| Povidon | 2.5 |
| Talcum | 0.1 |
| Microcrystalline cellulose (MCC) | 90.0 |
| Lactose | 110.0 |
| Magnesium stearate | 2.0 |
| Aerosil ® | 1.0 |
| Total | 416.1 |

Tablets comprising compounds as shown in Table 10 were prepared as described in Example 7.

### Example 9

### Pellet-Layering

TABLE 11

| | Amount in [mg] calculated |
|---|---|
| Ingredient | according to a single dose |
| Compound I micronized | 10 |
| Pellets (Suglets ®) | 200 |
| Sodium lauryl sulfate | 0.5 |
| Gum Arabica | 1.0 |
| Povidon | 2.5 |
| Talcum | 0.1 |
| Microcrystalline cellulose (MCC) | 90.0 |
| Lactose | 110.0 |
| Magnesium stearate | 2.0 |
| Aerosil ® | 1.0 |
| Total | 417.1 |

Tablets comprising compounds as shown in Table 10 were prepared as described in Example 4.

1. Pharmaceutical composition comprising
(a) a compound according to formula I as active ingredient 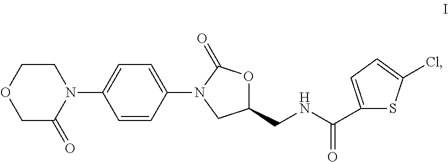
its solvates, hydrates and/or pharmaceutically acceptable salts,
(b) a solubilizer and
(c) a pseudo-emulsifier as excipients.

2. Pharmaceutical composition according to claim 1, wherein the composition comprises
(a) the compound according to formula I in crystalline form,
(b) cellulose or derivatives thereof or polyvinylpyrrolidone or copolymers thereof as solubilizer and
(c) a natural gum as pseudo-emulsifier.

3. Pharmaceutical composition according to claim 1, wherein the pseudo-emulsifier is present in an amount of 0.1 to 10 wt. %, based on the total weight of the composition.

4. Process for producing a pharmaceutical composition according to claim 1, comprising the steps of
(i) mixing a compound according to formula I and excipients,
(ii) dry-compaction of the mixture to give a comprimate, and
(iii) granulating the comprimate.

5. Process according to claim 4, wherein the dry-compaction step is carried out by roller compaction.

6. Process for producing a pharmaceutical composition according to claim 1, comprising the steps of
(i) providing a pellet core,
(ii) providing a solution or suspension comprising the compound according to formula I, and
(iii) spraying the solution or suspension onto the pellet core.

7. Process for producing a pharmaceutical composition according to claim 1, comprising the steps of:
(i) mixing a compound according to formula I and excipients
(ii) melting the mixture,
(iii) cooling off and granulating the melted mixture.

8. Process for producing a pharmaceutical composition according to claim 1, comprising the steps of:
(i) dissolving a compound according to formula I and polymer excipients in a solvent,
(ii) precipitating a complex comprising a compound according to formula I and polymer excipients by adding an anti-solvent, and
(iii) granulating the precipitated complex.

9. Granulates, obtainable by a process as described in claim 4.

10. Granulates according to claim 9, having a volume average particle size from 10 to 200 μm or from 250 to 650 μm.

11. Process for producing tablets comprising a pharmaceutical composition comprising the steps of:
(i) optionally mixing the granulates according to claim 9 with further excipients,
(ii) compressing the granulates according to claim 9 or the mixture of step (i) to give tablets,
wherein the pharmaceutical composition comprises
(a) a compound according to formula I as active ingredient 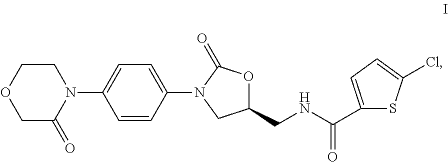
its solvates, hydrates and/or pharmaceutically acceptable salts,
(b) a solubilizer and
(c) a pseudo-emulsifier as excipients.

12. Process for producing tablets comprising a pharmaceutical composition according to claim 1, comprising the steps of:
(i) mixing a compound according to formula I and excipients.
(ii) direct-compressing the mixture.

13. Tablets obtainable by a process as described in claim 11.

14. Tablets according to claim 13, wherein the tablets are film-coated tablets for peroral use or dispersing tablets.

15. Pharmaceutical composition comprising:
(a) a compound according to formula I as active ingredient 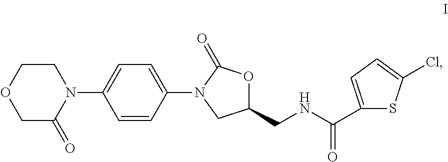
its solvates, hydrates and/or pharmaceutically acceptable salts, and
(b) a solubilizer,
obtainable by a process comprising the steps of
(i) providing a pellet core,
(ii) providing a solution or suspension comprising the compound according to formula (a) and a solubilizer (b), and
(iii) spraying, the solution or suspension onto the pellet core.

16. Granulates, obtainable by a process as described in claim 5.

17. Granulates, obtainable by a process as described in claim 6.

18. Granulates, obtainable by a process as described in claim 7.

19. Granulates, obtainable by a process as described in claim 8.

20. Tablets obtainable by a process as described in claim 12.
